(19) United States
(12) Patent Application Publication
(10) Pub. No.: US 2013/0317277 A1
(43) Pub. Date: Nov. 28, 2013

(54) RADIATION PROTECTION DEVICE FOR A SYRINGE

(71) Applicant: Pierre-Marie Lemer, Nantes (FR)

(72) Inventor: Pierre-Marie Lemer, Nantes (FR)

(73) Assignee: LEMER PROTECTION ANTI-X PAR ABREVIATION SOCIETE LEMER PAX, Carquefou (FR)

(21) Appl. No.: 13/984,402

(22) PCT Filed: Feb. 9, 2012
(86) PCT No.: PCT/FR2012/050284

§ 371 Date: Aug. 8, 2013

(30) Foreign Application Priority Data
Feb. 10, 2011 (FR) 1151088

(51) Int. Cl. A61N 5/10

(52) U.S. Cl. CPC A61N 5/1002
USPC 600/5

(57) ABSTRACT

A protection device to be fitted onto a syringe (2) used for injecting radioactive material(s). The radiation protection device includes a tubular radiation protection housing (1) that includes an outer tubular part (15), which constitutes at least a portion of the outer surface (11) thereof, and which is made of an elastomeric material. The tubular portion (15) also includes an annular constrictive portion (20) including a resiliently deformable inner surface that is used to apply a compressive force around the cylindrical body (3) of the inserted syringe (2).

The present invention relates to the general field of radiation protection. More precisely, it relates to the radiation protection devices of the “syringe shield” type, which are intended to be fitted onto the syringes used for injecting radioactive material(s) for the protection of the operators against the ionizing radiation.

Certain sectors of activity require the handling of radioactive materials, which emit ionizing radiation, such as radiation of electromagnetic nature (X, gamma) and/or particulate nature (alpha, beta, neutrons).

In the particular field of the nuclear medicine, radioactive materials are used for implementing diagnosis and/or treatment techniques, such as the in vivo functional imaging (for example, scintigraphy), the in vitro biological diagnosis (in particular in radioimmunology) and the metabolic radiotherapy.

The administration of such radioactive materials to the patient is often made by means of a syringe onto which is inserted a radiation protection device capable of attenuating the ionizing radiation, in particular to protect the operator handling this syringe.

Such a radiation protection device, commonly called “syringe shield”, is described for example in the documents EP-1 317 299 or U.S. Pat. No. 4,060,073.

This type of device comprises a radiation protection tubular housing that has—an inner surface, intended to cover at least a portion of the outer surface of the cylindrical body of the syringe, and—an outer surface, intended to be held in hand by the operator. This tubular housing comprises—a front opening, through which is intended to open out the front end of the cylindrical body of the syringe provided with a liquid suction and ejection orifice,—a rear opening, for the introduction and extraction of said cylindrical body of the syringe, and for the operation of its piston, and, often—a transparent screen made of radiation protective material, for visual access to the outer surface of the cylindrical body of the inserted syringe (to visualize the level of the front end of the piston within the syringe body).

When this tubular housing is entirely made of a metallic radiation protective material, for example lead or tungsten, this metallic structure generates drawbacks in different aspects.

Firstly, these metallic tubular housings are not completely effective to protect the inserted syringe against the shocks, in particular in case of fall. Therefore, such metallic housings do not offer an optimal solution for preventing the risks of syringe breakage. On the other hand, the appearance of these metallic tubular housings is generally not likely to reassure the patient (stressing effect) and these housings are often not much pleasant to touch.

Moreover, they are difficult to personalize and are not much attractive, in particular as regards the color. Therefore, it is sometimes tedious for the operators to identify the syringe shield that is adapted to syringe to be protected.

Another drawback is that the syringe body easily slips in the inner surface of the radiation protective housing, and that it may be necessary to provide complementary means for removably attaching this housing and the syringe to each other.

The document U.S. Pat. No. 4,060,073 provides a radiation protective housing formed of an inner shell made of rigid plastic material, covered with a cylindrical body made of a radiation protective material, itself covered with an outer shell made of rigid plastic material.

Here, the outer shell made of rigid plastic material does not protect efficiently the syringe against the shocks. Furthermore, the plastic inner shell is not adapted to efficiently prevent the syringe from slipping.

To make up for these drawbacks, the applicant has developed a new structure of syringe-shield device, which provides an efficient protection of the associated syringe against shocks, which offers an interesting perceived quality, an improved touch quality and a greater possibility of personalization, and which provides in a very simple way an efficient holding of the syringe.

The corresponding radiation protective device is of the type comprising a radiation protection tubular housing that has—an inner surface, intended to cover at least a portion of the outer surface of the cylindrical body of the syringe,—an outer surface,—a front opening, though which in intended to open out the front end of said cylindrical body, and—a rear opening, for the introduction and extraction of said cylindrical body, and for the operation of the piston.

And this tubular housing has also a tubular outer part, forming a portion at least of its outer surface (advantageously the whole or almost the whole of this housing outer surface), which is made of plastic material.
According to the invention, said tubular outer part is made of an elastomeric material, whose Shore A hardness value is advantageously comprised between 30 and 80; moreover, this outer part includes a constrictive annular portion comprising an inner surface that is resiliently deformable, said inner surface forming a section of the inner surface of the tubular housing and being intended to exert a clamping force around the cylindrical body of an inserted syringe.

Preferably, this constrictive annular portion forms the rear opening of the tubular housing.

According to another feature, this constrictive annular portion is advantageously capable of undergoing a resilient deformation between two configurations:
an active configuration, in which the inner surface is adapted to exert a clamping force around the cylindrical body of an inserted syringe, to generate a friction force opposing to the translation of the housing with respect to said cylindrical body, and
an inactive configuration, obtained by deformation (in particular, manual), in which its inner surface generates a reduced clamping force around said cylindrical body, to limit, or even cancel, the friction force opposing to the translation of the housing with respect to the syringe cylindrical body.

Within this framework, the inner surface of the housing preferably includes a section of circular cross-section defining a given diameter, which advantageously corresponds, to within the clearance, to the diameter of the cylindrical body of the inserted syringe and which extends the constrictive annular portion; and at rest, the inner surface of this constrictive annular portion defines an orifice of elongated shape, having, on the one hand, a small dimension that is lower than the diameter of said circular section of the housing and, on the other hand, a great dimension that is higher than the diameter of this circular section of the housing.

According to this embodiment, the constrictive annular portion advantageously includes, at its outer surface, two pressure areas that are arranged in a diametrically opposed manner and on a plane passing through the great dimension of the orifice of said constrictive annular portion, to facilitate the deformation of the latter in its inactive configuration.

The two pressure areas in question advantageously include a concave portion, for locally reducing the thickness of the constrictive annular portion and thus also reducing the force required for its deformation.

Preferably, the tubular housing includes a screen made of transparent radiation protective material, for visual access to the outer surface of the cylindrical body of the syringe.

According to still another feature, the tubular housing includes a tubular inner part made of radiation protective material, accommodated in the outer part and intended to form a portion at least of the inner surface of said housing.

This tubular inner part advantageously includes, over a portion at least of its length, a longitudinal opening opposite which is positioned the above-mentioned transparent screen made of radiation protective material.
The corresponding lateral opening is preferably delimited by two longitudinal edges which are each provided with a rebate for the nesting of the inner surface of the transparent screen.

The outer part advantageously includes an accommodation for the transparent screen, and if need be, an accommodation for the inner part, for the assembling thereof without complementary attaching means.

In this case, the accommodation for receiving the transparent screen made of radiation protective material is advantageously delimited by two opposite longitudinal walls that are connected by two transverse end walls, and, on the side of the outer surface of the housing, by an outer wall provided with a window enabling a visual access to said transparent screen.

Besides, the outer surface of the housing is advantageously provided with lateral recesses for optimizing the holding in hand of the device, said lateral recesses being diametrically opposed to each other and arranged on either side of the transparent radiation protection screen.

Moreover, the outer part made of elastomeric plastic material advantageously includes, on the side of the front opening of the tubular housing and at the opposite of the transparent screen, a generally bevel-shaped local truncation, to facilitate the positioning of the front end of an inserted syringe with respect to the point of injection of a patient.

The invention will be illustrated in more detail, without being limited in anyway, by the following description of a particular embodiment, in relation with the appended drawings, in which:

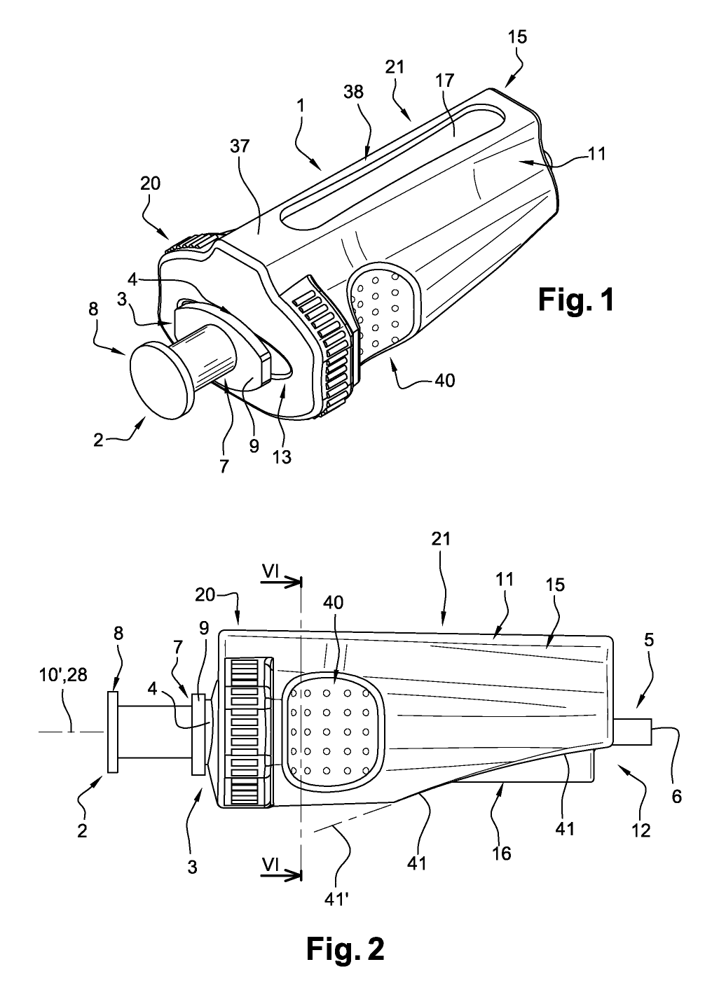

FIG. 1 is a general, perspective view of the radiation protection device, inserted onto a syringe;

FIG. 2 shows a side view of the radiation protection device and the syringe associated thereto;

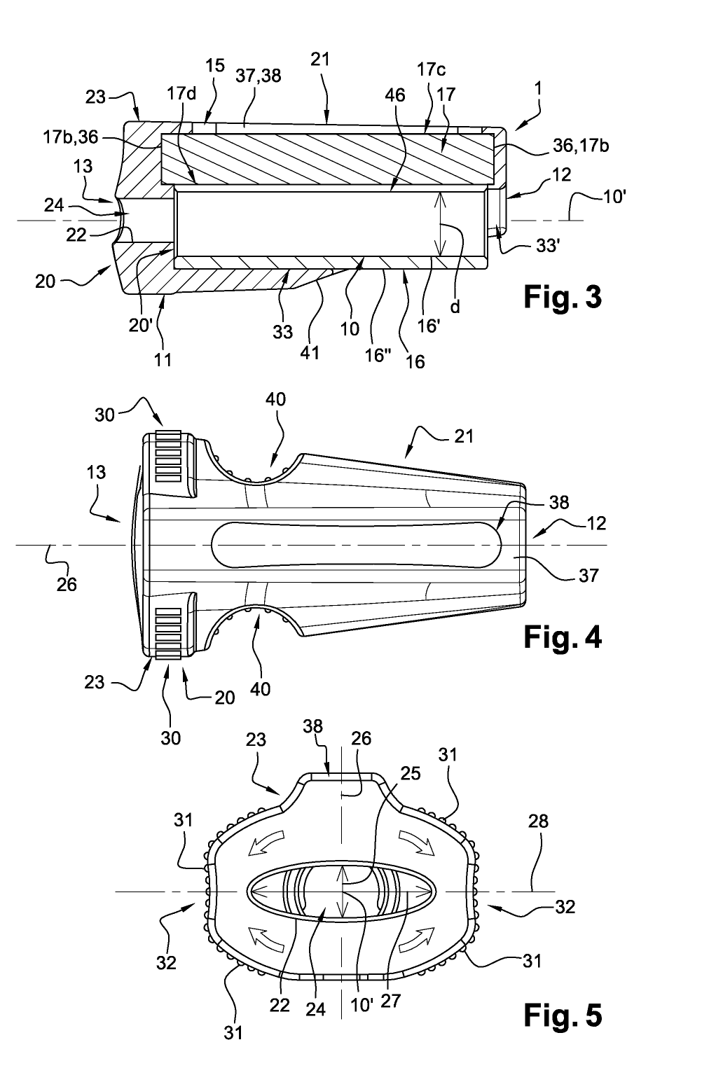

FIG. 3 is a sectional view of the radiation protection device illustrated in FIGS. 1 and 2, according to a longitudinal section plane passing through the transparent screen thereof;

FIG. 4 shows a top view of the radiation protection device;

FIG. 5 is a rear end view of the radiation protection device illustrated in FIG. 4;

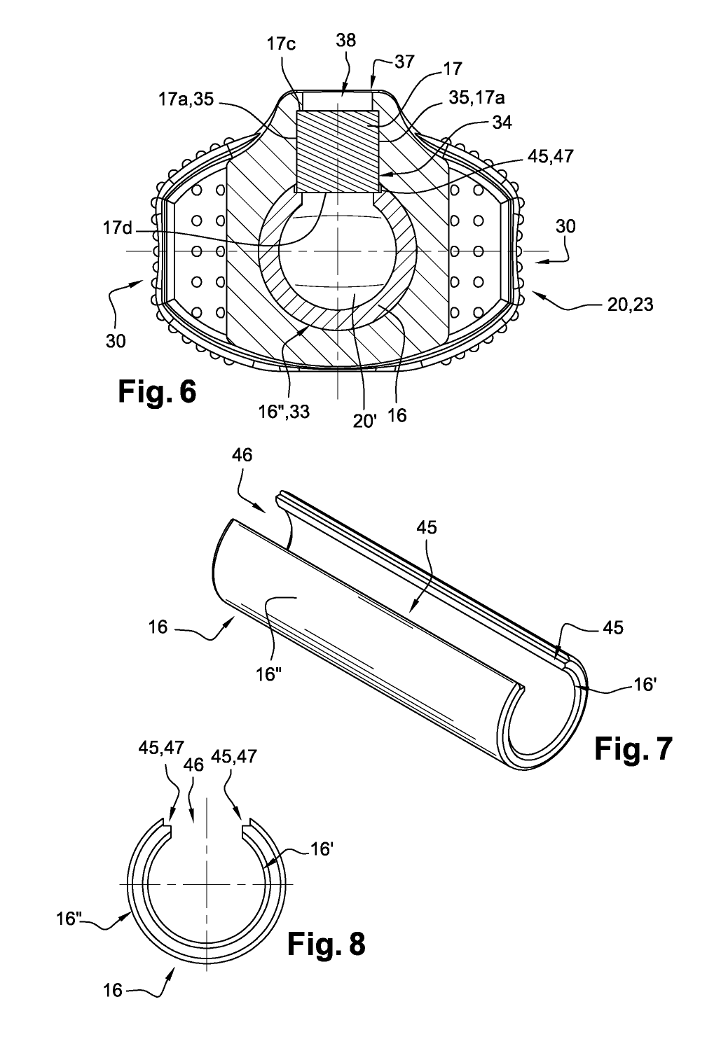

FIG. 6 is a transverse sectional view of the radiation protection device, according to the section plane VI-VI of FIG. 2;

FIG. 7 is a perspective view of the tubular inner part constitutive of the radiation protection device;

FIG. 8 is an end view of the tubular inner part according to FIG. 7.

As illustrated in FIGS. 1 and 2, the radiation protection device 1 is intended to be inserted onto a syringe 2 for injecting radioactive material(s).

Conventionally, the syringe 2 has a cylindrical body 3 provided with—an annular outer surface 4,—a front end 5, with a liquid suction and ejection orifice 6, and—a rear end 7, at which is inserted a piston 8 and which is provided with a rear flange 9.

The radiation protection device 1, also called “syringe shield”, consists in a tubular radiation protection housing that is inserted by fitting around the annular outer surface 4 of the cylindrical body 3 of the syringe 2.

This syringe-shield device 1 has the function of attenuating the ionizing radiation emitted by the liquid sucked into the body 3 of the syringe 2, and it thus protects the operators (as well as the patients) against the ionizing radiation emitted by this liquid.

The tubular housing 1 has two opposite surfaces 10 and 11, i.e.:
an inner surface 10, defining a longitudinal axis 10′ and intended to cover at least a portion of the length of the outer surface 4 of the cylindrical body 3 of the syringe 2 (FIG. 3), and
an outer surface 11, intended to be held in hand by the operator when the latter handles the syringe 2.

This tubular housing 1 has two openings 12 and 13, at which opens out the inner surface 10, i.e.:
a front opening 12, through which opens out the front end 5 of the cylindrical body 3 of the syringe 2, and
a rear opening 13, for the introduction and extraction of said cylindrical body 3, and for the axial operation of the piston 8.

In the embodiment illustrated, the tubular housing 1 is consisted of three parts 15, 16 and 17, which are assembled by nesting of complementary shapes, i.e.:
an outer part 15 made of elastomeric material, generally tubular in shape, forming in particular the outer surface 11 of the tubular housing 1,
an inner part 16 made of metallic radiation protective material, also generally tubular in shape, accommodated in said outer part 15 and having an inner surface 16′ forming a circular section of the inner surface 10 of the tubular housing 1, whose diameter is designated by the reference letter d (FIG. 3), and
a transparent screen 17, made of radiation protective material, for visual access to the cylindrical body 3 of the syringe.

The outer part 15 is in the form of a sheath, which forms here almost the whole of the outer surface 11 of the tubular housing 1.

This outer part 15 is made of an elastomeric material (advantageously silicone), whose Shore A hardness value is advantageously comprised between 30 and 80, preferably of the order of 40 to 50, and still preferably of the order of 45.

This tubular outer part 15 is advantageously obtained single-piece through an injection molding process.

Structurally, the outer part 15 may be divided into two portions 20 and 21, i.e.:
a constrictive annular portion 20, located on the side of the rear opening 13 of the tubular housing 1, and
a front annular portion 21, at which are inserted the inner part 16 and the transparent screen 17.

The constrictive annular portion 20 has two surfaces 22 and 23:
an inner surface 22, forming a rear section of the inner surface 10 of the tubular housing 1 (FIGS. 3 and 5), and
an outer surface 23, forming a rear section of the outer surface 11 of this same tubular housing 1.

The inner surface 22 of the constrictive annular portion 20 is resiliently deformable, so as to exert a clamping force around the cylindrical body 3 of the inserted syringe 2 (FIGS. 1 and 2).

For that purpose, as illustrated in FIG. 5, this inner surface 22 delimits here an orifice 24, of generally elongated shape, looking like an elongated oblong or oval shape.

The corresponding orifice 24 may be defined by two radial dimensions, square with respect to each other: the one 25, of small dimension, extending in a first radial plane of symmetry 26 passing through the radiation protection screen 17, and the other 27, of great dimension, passing through a second radial plane of symmetry 28 (perpendicular to the above-mentioned first plane 26).

To allow optimal deformation and clamping action, it is advantageously provided, on the one hand, that the small dimension 25 is lower than the diameter d of the circular section 16′ of the housing 1 defined by the inner part 16, and on the other hand, that the great dimension 27 is higher than this diameter d.

By way of indication, the small dimension 25 has a ratio comprised between 0.9 and 0.5 with respect to the diameter d, and the great dimension 27 has a ratio comprised between 1.5 and 2 with respect to the diameter d.

To facilitate the deformation of this constrictive annular portion 20, its outer surface 23 here includes two pressure areas 30 that are arranged in a diametrically opposed manner and on the plane of symmetry 28 passing through the great dimension 27 of the orifice 24.

The pressure areas 30 are each provided with a plurality of ribs or ridges 31, juxtaposed and oriented parallel to the longitudinal axis 10′, to optimize the positioning of the fingers of the operator.

They also each include a concave central portion 32, centered on the plane 28 passing through the great dimension 27 of the orifice 24, to further facilitate the holding by the fingers of the operator, and to reduce locally the thickness of this constrictive annular portion (FIG. 5).
This characteristic allows in particular to limit the force required for the deformation of this constrictive annular portion 20.

In operation, the constrictive annular portion 20 is thus capable of undergoing an resilient deformation between two configurations:
an active configuration, in which its inner surface 22 is capable of exerting a clamping force around the cylindrical body 3 of an inserted syringe 1, to generate a friction force opposing to the translation of the tubular housing 1 with respect to said cylindrical body 3 (FIGS. 1 and 2), and
an inactive configuration, obtained by a deformation of said constrictive annular portion 20, in which its inner surface 22 generates a reduced clamping force around said cylindrical body 3, to limit, or even cancel, the friction force opposing to the translation of the tubular housing 1 with respect to the cylindrical body 3 (not shown).

In the above-mentioned active configuration, the syringe 2 is suitably held within the radiation protection device 1, by the clamping of the annular inner surface 22 of the constrictive portion 20 (whereas the surface 16′ of the metallic inner part 16 exerts almost no clamping action on the syringe).

In the embodiment illustrated, the inactive configuration is obtained by a manual squeezing of the two pressure areas 30, toward each other.

This squeezing action generates a reduction of the value of the great dimension 27 and, simultaneously, an increase of the value of the small dimension 25. The orifice 24 thus takes a generally circular shape, which reduces, or even cancel, the contact force between the inner surface 22 of the constrictive portion 20 and the cylindrical body 3 of the syringe 2. This syringe 2 may then be easily removed from its protective housing 1.

On the other hand, as illustrated in FIGS. 3 and 6, the front portion 21 of the outer part 15 includes an inner surface that can be divided into two portions, i.e.:
a lower portion 33 having an arc of a circle cross-section, forming an accommodation intended to receive the tubular inner part 16, and
an upper portion 34, forming an accommodation receiving the radiation protection transparent screen 17.

In its receiving accommodation 33, the inner part 16 is here locked in translation by two stop surfaces (FIG. 3).

For that purpose, this accommodation 33 is provided, on the side of the front opening 12, with a protruding rib 33′ forming a front stop surface. And at the other end, the constrictive annular portion 20 includes a front surface 20′ defining a rear stop surface.

The reception housing 34 for the transparent screen 17 is arranged in the continuation of the lower housing 33; it is generally parallelepipedal in shape, with—two opposite longitudinal walls 35, each extending the lower part 33,—two transverse end walls 36, connecting said longitudinal walls 35, and—an outer wall 37 provided with a window or viewport 38 for a visual access to the radiation protection transparent screen 17.

On the side of the rear end of its outer surface 11, the front portion 21 of the outer part 15 still includes two concave lateral recesses 40, to optimize the holding in hand of the tubular housing 1 and of the associated syringe 2 (FIG. 4).

These lateral recesses 40 are diametrically opposed to each other and arranged on either side of the radiation protection transparent screen 17; they each have an arc of a circle cross-section, whose axis extends parallel to the plane of symmetry 26 passing through the transparent screen 17 (FIG. 4).

Moreover, on the side of its front end and at the opposite of the radiation protection transparent screen 17, the front portion 21 of the outer part 15 still includes a generally bevel-shaped local truncation 41, visible in FIGS. 2 and 3.

More precisely, this local truncation 41 extends in a plane 41′ defining an angle of the order of 15° with the plane of symmetry 28 perpendicular to the plane of symmetry 26 passing through the transparent screen 17; it may have an arc of a circle shape, with a rather small deflection.
The plane 41′ of this local truncation 41 passes, at the front opening 12, through a line corresponding at least approximately to the diameter of said opening 12. This truncation 41 ends, at least approximately, at half the length of this front portion 21.

This truncation 41 makes it easier to position the front end of the inserted syringe 2, with respect to the point of injection of patient.

In FIGS. 2 and 3, the inner part 16 protrudes with respect to the plane 41′, so that a front portion of its outer surface 16″ forms a portion of the outer surface 11 of the tubular housing 1.

By way of indication, the thickness of the constrictive annular portion 20 (corresponding to the distance between its inner 22 and outer 23 surfaces) is advantageously comprised between 0.3 and 1 cm. The thickness of the front portion 21 of the outer part 15 (corresponding to the distance between its inner 33 and outer 11 surfaces) is advantageously comprised between 0.2 and 0.6 cm.

The inner portion 16, which is notably visible in FIGS. 3 and 6, is shown alone in FIGS. 7 and 8.

This tubular inner part 16 is made of one or several layers of radiation protective material, for example tungsten, lead, tantalum, filled compound, or more generally any material liable to attenuate the ionizing radiation.

It has a generally arc of a circle cross-section, with two opposed surfaces, an inner one 16′, forming the circular section of the inner surface 10 of the tubular housing 1, and an outer one 16″, whose diameter corresponds to the diameter defined by the dedicated accommodation 33 of the outer part 15.

These two opposed surfaces 16′ and 16″ join together at the two longitudinal edges 45, opposite to each other and remote from each other, which define a longitudinal opening 46 at which is intended to be positioned the radiation protection transparent screen 17 (FIGS. 3 and 6).

This longitudinal opening 46 here extends over the whole length of the inner part 16.

In order to optimize the positioning and the holding of the radiation protection transparent screen 17, the two above-mentioned longitudinal edges 45 are each provided with respective rebates 47, opposite to each other, for the positioning of the inner part of this transparent screen 17.

By way of indication, the thickness of this inner part 16 is constant and advantageously comprised between 0.5 and 15 mm, preferably between 1 and 3 mm.

The transparent screen 17 itself consists of a parallelepipedal rod or bar, whose dimensions correspond to those of the dedicated accommodation 34 of the outer part 15.

This screen 17 is made of a transparent radiation protective material, such as lead glass or a transparent plastic material filled with radiation attenuation elements.

This radiation protection transparent screen 17 has the following faces:
two lateral longitudinal faces 17a, conforming the lateral walls 35 of the dedicated accommodation 34,
two transverse walls 17b, conforming the transverse walls 36 of the accommodation 34,
an upper longitudinal wall 17c, conforming the upper wall 37 of the accommodation 34, and
a lower longitudinal wall 17d, conforming the two longitudinal edges 45 provided with the rebates 47 of the inner part 16.

By way of indication, the thickness of this screen 17 (corresponding to the distance between its upper 17c and lower 17d longitudinal walls) is advantageously comprised between 5 and 20 mm.

The angles of the screen 17 located between the lower wall 17d and the longitudinal walls 17a are positioned and locked in the rebates 47 of the inner part 16; and this unit is accommodated in the adapted accommodations 33 and 34 of the outer part 15.

The positioning of the inner part 16 and of the screen 17 within the outer part 15 is made thanks to the resilience of the latter; and this resilience provides the assembling of the three parts 15, 16 and 17, without requiring other attaching means.

The syringe-shield device according to the invention has the interest to be capable of being simply and rapidly assembled or disassembled, which is useful in particular for the operations of maintenance, repairing or cleaning of its different constitutive parts 15, 16 and 17.

In operation, when an operator wants to protect a syringe 2, he just has to choose and fit a radiation attenuation housing 1 adapted to the dimensions of the latter.

For that purpose, it is advantageously provided to propose a range of syringe-shield devices, whose dimensions are adapted to the different syringes to be equipped.

To simplify the choice of the syringe-shield device 1 adapted to the syringe 2 to be protected, the outer parts 15 of the range may have distinctive colors, which is made possible in a very simple way because they are made of plastic material.

To fit the chosen syringe-shield device 2, the operator applies a pressure on the pressure areas 30, toward each other; then, he introduces axially the front end 5 of the syringe 2 until the flange 9 thereof comes in abutment against the rear face of the constrictive annular portion 20.

The operator may then release the pressure areas 30, so that the constrictive annular portion 20 tighten automatically around the body 3 of the syringe 2.
He may then take and handle the radioactive material(s) to be injected to the patient by means of the protected syringe 2, by holding the tubular housing 1.

Due to the materials used (in particular the elastomer material), the outer part 15 offers an improved touch quality, with in particular a high friction force which reduces the risks of involuntary slipping of the syringe 2.

The volume of liquid taken in the syringe 2 may be directly viewed through the transparent screen 17.

If the unit syringe/syringe-shield were accidentally dropped, the outer part 15 absorbs efficiently the shocks, and the risks of deterioration (both of the syringe 2 and of the transparent screen 17) are then significantly reduced.

Then, to discard the syringe 2, the operator has just to exert a new pressure on the pressure areas 30 of the constrictive annular portion 20 and, while maintaining this pressure, to apply an axial traction on the syringe 2 (or to let the syringe slip by gravity) for separating it from the syringe-shield device.

By way of alternative, the outer part 15 may be made of radiation attenuation plastic material; in this case, the inner part 16 would then be no more really useful and could be suppressed.

1. A radiation protection device intended to be fitted onto a syringe (2) for injecting radioactive material(s), said syringe (2) having a cylindrical body (3) provided with—an outer surface (4),—a front end (5), including a liquid suction and ejection orifice (6), and—a rear end (7), at which is inserted a piston (8), said radiation protection device includes a radiation protection tubular housing (1) which has—an inner surface (10), intended to cover at least a portion of said outer surface (4) of the cylindrical body (3),—an outer surface (11),
a front opening (12), through which is intended to open out said front end (5) of said cylindrical body (3),—a rear opening (13), for the introduction and extraction of said cylindrical body (3), and for the operation of said piston (8), said housing (1) including a tubular outer part (15), forming a portion at least of its outer surface (11), which is made of plastic material, characterized in that said tubular outer part (15) is made of elastomeric material, and in that it includes a constrictive annular portion (20) comprising an inner surface (22) that is resiliently deformable, said inner surface (22) forming a section of the inner surface (10) of the housing (1) and being intended to exert a clamping force around the cylindrical body (3) of an inserted syringe (2).

2. The radiation protection device according to claim 1, characterized in that the elastomeric material forming the tubular outer part (15) has a Shore A hardness value comprised between 30 and 80.

3. The radiation protection device according to claim 1, characterized in that the constrictive annular portion (20) forms the rear opening (13) of the tubular housing (1).

4. The radiation protection device according to claim 1, characterized in that the constrictive annular portion (20) is capable of undergoing a resilient deformation between two configurations:
an active configuration, in which its inner surface (22) is adapted to exert a clamping force around the cylindrical body (3) of an inserted syringe (2), to generate a friction force opposing to the translation of the housing (1) with respect to said cylindrical body (3), and
an inactive configuration, obtained by deformation, in which its inner surface generates a reduced clamping force around said cylindrical body (3), to limit, or even cancel, the friction force opposing to the translation of the housing (1) with respect to said syringe cylindrical body (3).

5. The radiation protection device according to claim 4, characterized in that the inner surface (10) of the housing (1) includes a section (16′) of circular cross-section defining a determined diameter (d), extending the constrictive annular portion (20), and in that, at rest, the inner surface (22) of said constrictive annular portion (20) defines an orifice (24) of generally elongated shape, including, on the one hand, a small dimension (25) that is lower than the diameter (d) of said circular section (16′) of the housing (1) and, on the other hand, a great dimension (27) that is higher than said diameter (d).

6. The radiation protection device according to claim 5, characterized in that the constrictive annular portion (20) includes, at its outer surface (23), two pressure areas (30) that are arranged in a diametrically opposed manner and on a plane (28) passing through the great dimension (27) of the orifice (24) of said constrictive annular portion (20), to facilitate the deformation of the latter in its inactive configuration.

7. The radiation protection device according to claim 1, characterized in that the tubular housing (1) comprises a screen made of transparent radiation protective material (17), for visual access to the outer surface (4) of the cylindrical body (3) of the syringe (2).

8. The radiation protection device according to claim 7, characterized in that the housing (1) also includes a tubular inner part (16) made of radiation protective material, accommodated in the outer part (15) and intended to form a portion at least of the inner surface (10) of said housing (1).

9. The radiation protection device according to claim 8, characterized in that the tubular inner part (16) includes, over a portion at least of its length, a longitudinal opening (46) opposite which is positioned the transparent screen made of radiation protective material (17).

10. The radiation protection device according to claim 9, characterized in that the inner part (16) includes two longitudinal edges (45) delimiting the longitudinal opening (46), said longitudinal edges (46) being each provided with a rebate (47) for the nesting of the inner surface (17d) of the transparent screen (17).

11. The radiation protection device according to claim 7, characterized in that the outer part (15) includes an accommodation (34) for receiving the transparent screen (17), and if need be, an accommodation (33) for receiving the inner part (16), for the assembling thereof without complementary attaching means.

12. The radiation protection device according to claim 11, characterized in that the accommodation (34) for receiving the screen made of transparent radiation protective material (17) is delimited by two opposite longitudinal walls (35) which are connected by two transverse end walls (36), and, on the side of the outer surface (11) of the housing (1), by an outer wall (37) provided with a window (38) enabling a visual access to said transparent screen (17).

13. The radiation protection device according to claim 2, characterized in that the constrictive annular portion (20) forms the rear opening (13) of the tubular housing (1).

14. The radiation protection device according to claim 2, characterized in that the constrictive annular portion (20) is capable of undergoing a resilient deformation between two configurations:
an active configuration, in which its inner surface (22) is adapted to exert a clamping force around the cylindrical body (3) of an inserted syringe (2), to generate a friction force opposing to the translation of the housing (1) with respect to said cylindrical body (3), and
an inactive configuration, obtained by deformation, in which its inner surface generates a reduced clamping force around said cylindrical body (3), to limit, or even cancel, the friction force opposing to the translation of the housing (1) with respect to said syringe cylindrical body (3).

15. The radiation protection device according to claim 3, characterized in that the constrictive annular portion (20) is capable of undergoing a resilient deformation between two configurations:
an active configuration, in which its inner surface (22) is adapted to exert a clamping force around the cylindrical body (3) of an inserted syringe (2), to generate a friction force opposing to the translation of the housing (1) with respect to said cylindrical body (3), and
an inactive configuration, obtained by deformation, in which its inner surface generates a reduced clamping force around said cylindrical body (3), to limit, or even cancel, the friction force opposing to the translation of the housing (1) with respect to said syringe cylindrical body (3).

16. The radiation protection device according to claim 1, characterized in that the housing (1) also includes a tubular inner part (16) made of radiation protective material, accommodated in the outer part (15) and intended to form a portion at least of the inner surface (10) of said housing (1).
